(19) United States
(12) Patent Application Publication
(10) Pub. No.: US 2010/0260696 A1
(43) Pub. Date: Oct. 14, 2010

(54) Aqueous Compositions Containing Alkoxylated Phosphoric Acid Triesters

(75) Inventors: Peter Klug, Grossostheim (DE); Franz-Xaver Scherl, Burgkirchen (DE); Waltraud Simsch, Kelkheim (DE); Adelgunde Oberhauser, Neuoetting (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT FINANCE (BVI) LIMITED, Tortola (VG)

(21) Appl. No.: 12/671,441

(22) PCT Filed: Jul. 29, 2008
(86) PCT No.: PCT/EP08/06219

§ 371 Date: Jun. 22, 2010

(30) Foreign Application Priority Data
Aug. 2, 2007 (DE) 10 2007 036 184.1
Jan. 31, 2008 (DE) 10 2008 006 858.6
Jul. 29, 2008 (EP) PCT/EP2008/006219

(51) Int. Cl. A61K 8/55; A61K 47/24; A61Q 15/00; A61P 17/00; A61P 43/00

(52) U.S. Cl. 424/65; 514/772

(57) ABSTRACT

The invention relates to compositions containing A) at least one phosphoric acid ester of formula (I)

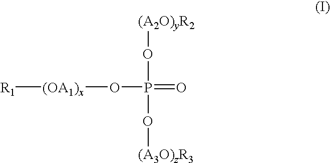

wherein R₁, R₂ and R₃ can be the same or different and stand for a linear or branched, saturated alkyl group comprising between 6 and 30 carbon atoms, a linear or branched, monounsaturated or polyunsaturated alkenyl group comprising between 6 and 30 carbon atoms, or an aryl group that can be substituted with between 1 and 3 branched alkyl groups each independently containing between 3 and 18 carbon atoms, the individual groups (OA₁)ₓ, (A₂O)_{y} and (A₃O)_{z} each independently consisting of units selected from CH₂CH₂O, C₃H₆O and C₄H₈O, the units CH₂CH₂O, C₃H₆O and C₄H₈O being arranged inside the individual groups (OA₁)X, (A₂O)_{y} and (A₃O), in blocks or in a statistically distributed manner, and x, y and z each independently standing for a number between 30 and 150, and B); water in a proportion of >50.0 wt. % in relation to the finished composition. The compositions according to the invention are preferably cosmetic, pharmaceutical or dermatological compositions.

This invention relates to aqueous compositions containing alkoxylated phosphoric triesters and also to the use of these phosphoric triesters as thickeners or associative thickeners, particularly in cosmetic, pharmaceutical or dermatological compositions.

Cosmetic products have to meet high requirements. They shall have a clear appearance, be generally recognized as safe by toxicologists and ecotoxicologists, create a pleasant skin feel and have excellent rheological behavior which is constant over a wide pH range.

Water- or solvent-containing multicomponent systems such as emulsions or suspensions are frequently adjusted to higher viscosities, i.e., thickened, for economic reasons, for performance reasons or for stability reasons.

For instance, increasing the viscosity of the external or internal phase of emulsions or suspensions lengthens the time to separation of the components of such a system distinctly, which manifests itself in a lengthening of the storage time. Increasing the viscosity also improves for many products their uniform distributability on nonplanar surfaces in particular.

The more uniform distribution and lengthened active time enhances the efficacy. In addition to the performance advantages mentioned, the high viscosity of such products also offers further advantages in relation to manufacture, packaging, filling and storage and also in transportation.

The technical literature contains reports of a large number of different systems for adjusting the rheological properties of aqueous or solvent-containing systems, emulsions or suspensions. Known examples are cellulose ethers and other cellulose derivatives (for example carboxymethylcellulose, hydroxyethylcellulose), gelatin, starch and starch derivatives, sodium alginates, fatty acid polyethylene glycol esters, agar, tragacanth or dextrins. By way of synthetic polymers, various materials are used, examples being polyvinyl alcohols, polyacrylamides, polyacrylic acid and various salts of polyacrylic acid, polyvinylpyrrolidone, polyvinyl methyl ether, polyethylene oxides, copolymers of maleic anhydride and vinyl methyl ether, and also diverse mixtures and copolymers thereof.

However, the compounds mentioned display manifold disadvantages in use. For instance, cellulose derivatives and, in general, materials based on natural raw materials and the formulations resulting therefrom are very vulnerable to bacteria. Technically, they usually form unpleasant, “ropey” gels.

Fatty acid polyethylene glycol esters tend to hydrolyze in the presence of water and the resulting insoluble fatty acids cause undesirable clouding. Thickeners of natural origin (for example agar or tragacanth) fluctuate substantially in composition, depending on provenience.

Cosmetic preparations comprising long-chain mono- and diphosphoric esters are described in DE 10 2004 047 092, U.S. Pat. No. 6,448 297 and DE 10 2004 046 356 and utilize the emulsifying effect of phosphoric esters. A sufficiently thickening effect on the part of the phosphoric esters is not observed.

JP 09268193 describes a process for preparing alkoxylated phosphoric triesters having 1 to 50, preferably 1 to 10 alkoxy groups and points to their use in cosmetic products which advantageously utilize the skin-friendly properties of the triesters and also their low viscosities and low melting points. There are no pointers to a thickening effect of aqueous-based formulations.

U.S. Pat. No. 5,129,462 describes shampoo formulations comprising polyethylene glycol polyol fatty acid esters, particularly PEG pentaerythritol fatty acid esters as thickeners. The processing and formulatability of this class of compounds is impaired by their high melting points or setting points.

EP 1 518 900 and EP 1 344 518 disclose cosmetic and pharmaceutical preparations comprising oxyalkylated polyglycerol esters as thickeners, dispersants for aqueous, aqueous-alcoholic and aqueous-surfactant preparations and as emulsifiers, suspending agents having a thickening effect and consistency regulators for emulsions and suspensions.

The associative thickeners described in the references U.S. Pat. No. 5,129,462, EP 1 518 900 and EP 1 344 518 cited still have room for improvement with regard to their thickening performance, specifically in purely aqueous systems, where they only form cloudy gels, but also with regard to their stability at low pH. At below pH 5 their gels and thickened surfactant solutions are not stable in storage, but very rapidly lose viscosity.

It is an object of the present invention to provide a novel class of substances which is suitable as a thickener for aqueous compositions, in particular for cosmetic, dermatological or pharmaceutical aqueous compositions, which gives the formulations a clear appearance and which even in a very acidic medium or at high electrolyte contents engenders a high thickening capacity in the event of thermal stress and following long storage times.

We have found that this object is achieved, surprisingly, by the alkoxylated phosphoric esters of formula (I) described below, which represent alkoxylated phosphoric triesters.

The present invention accordingly provides compositions comprising
A) one or more phosphoric esters of formula (I)

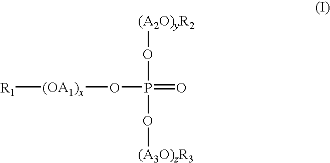

where
R₁, R₂ and R₃ may be identical or different and each is a linear or branched saturated alkyl group having 6 to 30, preferably 8 to 22 and more preferably 12 to 18 carbon atoms, is a linear or branched mono- or polyunsaturated alkenyl group having 6 to 30, preferably 8 to 22 and more preferably 12 to 18 carbon atoms, or is an aryl group, more particularly a phenyl group, which may be substituted with 1 to 3 branched alkyl groups which each independently comprise 3 to 18 and preferably 4 to 12 carbon atoms,
the individual groups (OA₁)ₓ, (A₂O)_{y} and (A₃O)_{z} each independently consist of units selected from CH₂CH₂O, C₃H₆O and C₄H₈O and the units CH₂CH₂O, C₃H₆O and C₄H₈O may be arranged blocklike or randomly distributed within the individual groups (OA₁)ₓ, (A₂O)_{y} and (A₃O)_{z}, and
x, y and z are each independently a number from 30 to 150, preferably from 40 to 120 and more preferably from 51 to 100, and
B) water in an amount >50.0% by weight, preferably in an amount >70.0% by weight and more preferably in an amount of 75.0% to 95.0% by weight, based on the final composition.

The phosphoric triesters of formula (I) are preferably obtainable by reaction of phosphoric acid or phosphoric acid derivatives with alkoxylated fatty alcohols characterized in that they bear at least 30 alkoxy groups.

In one preferred embodiment of the present invention, the radicals R₁, R₂ and R₃ in the phosphoric esters of formula (I) may be identical or different and each is a linear or branched saturated alkyl group having 6 to 30, preferably 8 to 22 and more preferably 12 to 18 carbon atoms, or is a linear or branched mono- or polyunsaturated alkenyl group having 6 to 30, preferably 8 to 22 and more preferably 12 to 18 carbon atoms;

In a further preferred embodiment of the present invention, the units OA₁, OA₂ and OA₃ in the phosphoric esters of formula (I) are each CH₂CH₂O.

In a particularly preferred embodiment of the present invention, the radicals R₁—(OA₁)ₓ, R₂—(OA₂)_{y} and R₃—(OA₃)_{z} in the phosphoric esters of f derived from fatty alcohol ethoxylates selected from C_{16/18} fatty alcohol ethoxylates having 30 to 150 ethylene oxide units, preferably having 40 to 120 ethylene oxide units, more preferably C_{16/18} fatty alcohol ethoxylate having 50 ethylene oxide units (for example Genapol® T 500) or C_{16/18} fatty alcohol ethoxylate having 80 ethylene oxide units (for example Genapol® T 800).

In a further preferred embodiment of the present invention, the groups (OA₁)ₓ, (OA₂)_{y} and (OA₃)_{z} in the phosphoric esters of formula (I) are each constructed of CH₂CH₂O and C₃H₆O units, which CH₂CH₂O and C₃H₆O units may be arranged blocklike or randomly distributed within the individual groups (OA₁)ₓ, (OA₂)_{y} and (OA₃)_{z} and each comprise 30 to 150, preferably 40 to 120 and more preferably 51 to 100 CH₂CH₂O units and 0 to 20, preferably 1 to 10 and more preferably 2 to 5 C₃H₆O units.

In a further particularly preferred embodiment of the present invention, the radicals R₁—(OA₁)ₓ, R₂—(OA₂)_{y} and R₃—(OA₃)_{z} in the phosphoric esters of formula (I) are derived from fatty alcohol ethoxylate-propoxylate selected from C_{16/18} fatty alcohol ethoxylate-propoxylate having 50 ethylene oxide units and 2 propylene oxide units or C_{16/18} fatty alcohol ethoxylate-propoxylate having 50 ethylene oxide units and 5 propylene oxide units.

The phosphoric esters in the compositions of the present invention may also constitute mixtures of phosphoric esters, in which case the proportion attributable to phosphoric triesters is then preferably more than 80% by weight, based on the phosphoric ester mixture.

In a further preferred embodiment of the present invention, the compositions of the present invention, in addition to the one or more phosphoric esters of formula (I), comprise one or more phosphoric esters of formula (II)

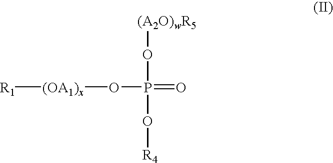

where
R₁ is a linear or branched saturated alkyl group having 6 to 30, preferably 8 to 22 and more preferably 12 to 18 carbon atoms, is a linear or branched mono- or polyunsaturated alkenyl group having 6 to 30, preferably 8 to 22 and more preferably 12 to 18 carbon atoms, or is an aryl group, more particularly a phenyl group, which may be substituted with 1 to 3 branched alkyl groups which each independently comprise 3 to 18 and preferably 4 to 12 carbon atoms, R₄ is H, Li⁺, Na⁺, K⁺, Mg⁺⁺, Ca⁺⁺, Al⁺⁺⁺, NH₄⁺ or quaternary ammonium ions [HNR^{a}R^{b}R^{c}]⁺, in which R^{a}, R^{b} and R^{e} independently stand for hydrogen, a linear or branched alkyl group having 1 to 22 carbon atoms, a linear or branched mono- or polyunsaturated alkenyl group having 2 to 22 carbon atoms, a linear monohydroxyalkyl group having 2 to 10 carbon atoms, preferably a monohydroxyethyl or monohydroxypropyl group, or a linear or branched dihydroxyalkyl group having 3 to 10 carbon atoms.
R₅ has the meaning of R₁ or R₄,
the individual groups (OA₁)ₓ and (A₂O)_{w} each independently consist of units selected from CH₂CH₂O, C₃H₆O and C₄H₈O and the units CH₂CH₂O, C₃H₆O and C₄H₈O may be arranged blocklike or randomly distributed within the individual groups (OA₁)ₓ and (A₂O)_{w},
x is a number from 30 to 150, preferably from 40 to 120 and more preferably from 51 to 100, and
w is 0 or is a number from 30 to 150, preferably from 40 to 120 and more preferably from 51 to 100,
with the proviso that the amount of phosphoric triesters as per formula (I) is greater than 80.0% by weight, preferably in the range from 85.0% to 99.0% by weight and more preferably in the range from 88.0% to 95.0% by weight, based on the total weight of the phosphoric esters as per formula (I) and formula (II), and the degree of neutralization of the nonesterified phosphorus valences (P—OH) in the phosphoric esters as per formula (II) may be between 0 and 100%.

In the phosphoric esters of formula (II) R¹ is preferably a linear or branched, saturated alkyl group having 6 to 30, preferably 8 to 22 and more preferably 12 to 18 carbon atoms or is a linear or branched, mono- or polyunsaturated alkenyl group having 6 to 30, preferably 8 to 22 and more preferably 12 to 18 carbon atoms. The same applies to R⁵ when it has the meaning of R¹. In this case however R¹ and R⁵ may be identical or different.

In the compounds of formula (II) (OA₁)ₓ where w=0 or (OA₁)ₓ and (A₂O)_{w} where w≠0 preferably consist of units selected from CH₂CH₂O and C₃H₆O and particularly preferably of CH₂CH₂O units.

The phosphoric esters of formula (I) or mixtures of the phosphoric esters of formulae (I) and (II) are preferably prepared by reacting phosphoric acid or their derivatives with fatty alcohol alkoxylates at temperatures between 150 and 250° C., preferably between 180 and 240° C. and more preferably between 200 and 230° C.

Preferred phosphoric acid derivatives are selected from polyphosphoric acid, tetraphosphorus decaoxide, phosphoryl chloride and phosphorus pentachloride.

Orthophosphoric acid is the preferred reactant.

As fatty alcohol alkoxylate reactants preference is given to fatty alcohol ethoxylates, more preferably fatty alcohol ethoxylates having 30 to 150 EO units (EO═CH₂CH₂O), even more preferably having 40 to 120 EO units and yet even more preferably having 51 to 100 EO units, the respective fatty alcohol radical (R₁O—, R₂O— and/or R₃O—) being derived from alcohols selected from octanol, decanol, dodecanol, tetradecanol, hexadecanol, octadecanol, eicosanol, behenyl alcohol, fatty alcohols having C-chain cuts between 8 and 22, preferably C₁₀/C₁₂ fatty alcohol, C₁₂/C₁₄ fatty alcohol, C₁₂/C₁₅ fatty alcohol and C₁₆/C₁₈ fatty alcohol, branched fatty alcohols, preferably Guerbet alcohols and monounsaturated fatty alcohols, preferably delta-9-cis-hexadecanol, delta-9-cis-octadecanol, trans-9-octadecanol and cis-delta-11-octadecanol.

Further preferred fatty alcohol alkoxylate reactants are fatty alcohol ethoxy propoxylates, more preferably fatty alcohol ethoxy propoxylates having 30 to 150 CH₂CH₂O units (EO) and 0 to 20 C₃H₆O units (PO), more preferably having 40 to 120 EO and 2 to 10 PO units and even more preferably having 51 to 100 EO and 2 to 5 PO units, wherein the fatty alcohol radicals are derived from the abovementioned fatty alcohols.

The esterification is preferably carried on such that essentially neutral phosphoric triesters are present. Preference is given to a degree of conversion >80%, i.e., more than 80% of all esterifiable functions of the phosphoric acid or phosphoric acid derivatives are esterified. A degree of conversion >90% is particularly preferred and >95% even more particularly preferred.

The remaining free valences on the phosphorus atom can be acid groups, but also counter ions selected from Li⁺, Na⁺, K⁺, Mg⁺⁺, Ca⁺⁺, Ar⁺⁺⁺, NH₄⁺ and quaternary ammonium ions [HNR^{a}R^{b}R^{c}]⁺, in which R^{a}, R^{b} and R^{c} independently can be hydrogen, a linear or branched alkyl group having 1 to 22 carbon atoms, a linear or branched mono- or polyunsaturated alkenyl group having 2 to 22 carbon atoms, a linear monohydroxyalkyl group having 2 to 10 carbon atoms, preferably a monohydroxyethyl or monohydroxypropyl group, and also a linear or branched dihydroxyalkyl group having 3 to 10 carbon atoms.

The degree of neutralization of the unsubstituted phosphorus valences (P-OH) can be between 0% and 100%.

In one preferred embodiment of the present invention, the phosphoric esters of formula (II) in the compositions of the present invention are neutralized to a degree of neutralization of 0-20.0%.

In another preferred embodiment of the present invention, the phosphoric esters of formula (II) in the compositions of the present invention are neutralized to a degree of neutralization of 20.1-100%.

The compositions of the present invention may comprise one or more phosphoric esters of formula (II) where R⁵ has the meaning of R⁴ and w is 0. These compounds comprise monophosphoric esters.

However, the compositions of the present invention preferably comprise such compounds of formula (II) where R₅ has the meaning of R₁ and w is a number from 30 to 150, preferably from 40 to 120 and more preferably from 51 to 100. These compounds comprise diphosphoric esters.

The compositions of the present invention may comprise mixtures of monophosphoric esters and diphosphoric esters of formula (II).

When the compositions of the present invention comprise monophosphoric esters of formula (II), the amount thereof is preferably <5.0% by weight, more preferably <3.0% by weight, even more preferably <1.0% by weight and yet even more preferably <0.1% by weight, based on the final composition.

In a further preferred embodiment of the present invention, the compositions of the present invention comprise water in an amount >90.0% by weight and preferably in an amount of 90.1% to 95.0% by weight, based on the final composition.

The phosphoric esters of formula (I) have excellent thickening capacity not only for compositions on an aqueous or aqueous-alcoholic basis but also for compositions on an aqueous-surfactant basis and tolerate even organic solvents such as alcohols.

In the compositions of the present invention, the amounts in which the one or more phosphoric esters of formula (I) are present are preferably in the range from 0.1% to 10.0% by weight, more preferably in the range from 0.5% to 8.0% by weight and even more preferably in the range from 1.0% to 6.0% by weight, based on the final compositions.

The aqueous compositions of the present invention have viscosities of 100 to 100 000 mPa·s, preferably of 1000 to 50 000 mPa·s and more preferably of 2000 to 25 000 mPa·s at room temperature.

The phosphoric esters of formula (I) are very useful for thickening aqueous-surfactant compositions.

In a further preferred embodiment of the present invention, the compositions of the present invention comprise one or more surfactants. Specifically these surfactant-containing compositions of the present invention preferably comprise the one or more phosphoric esters of formula (I) in amounts of 0.1% to 5.0% by weight, more preferably 0.2% to 4.0% by weight and even more preferably 0.5% to 3.0% by weight, based on the final surfactant-containing compositions.

The aqueous-surfactant compositions of the present invention have viscosities of 100 to 50 000 mPa·s, preferably of 500 to 25 000 mPa·s and more preferably of 1000 to 10 000 mPa·s at room temperature.

The phosphoric esters in the compositions of the present invention are notable for good skin compatibility and are highly suitable for use in cosmetic, dermatological and pharmaceutical compositions.

In a further preferred embodiment of the present invention, the compositions of the present invention are therefore cosmetic, dermatological or pharmaceutical compositions.

The phosphoric esters of formula (I) have manifold possible uses and are suitable for use in aqueous, aqueous-alcoholic and aqueous-surfactant compositions, emulsions, suspensions, dispersions, powders and sprays.

In a further preferred embodiment of the invention the compositions of the present invention are therefore present in the form of aqueous, aqueous-alcoholic or aqueous-surfactant compositions, in the form of emulsions, suspensions, dispersions or sprays.

It is also particularly advantageous that the thickening capacity is also marked in a strong acidic medium.

The phosphoric esters of formula (I) are therefore particularly useful for thickening and stabilizing acidic cosmetic compositions. These can be for example cosmetic compositions comprising hydroxyacids, such as lactic acid, glycolic acid, salicylic acid, citric acid or polyglycol diacids in free form or partial neutralization. It is further possible to stabilize formulations comprising vitamin C or vitamin C derivatives, dihydroxyacetone or skin-whitening actives such as arbutin or glycyrrhetic acid and salts thereof.

In a further preferred embodiment of the present invention, the compositions of the present invention have a pH in the range from 2 to 10, preferably in the range from 2 to 6, more preferably in the range from 2.5 to 5 and even more preferably in the range from 3 to 4.5.

Particular preference is given to compositions of the present invention with a pH from 2.5 to 5 which, based on the entire composition, comprise from 0.05% to 3.0% by weight, preferably from 0.05% to 2.0% by weight and more preferably from 0.1% to 1.0% by weight of one or more organic acids having an antimicrobial effect, preferably carboxylic acids having an antimicrobial effect. Particularly preferred organic acids having an antimicrobial effect are benzoic acid, sorbic acid, salicylic acid, lactic acid and anissic acid. These acids can also be used in the compositions of the present invention, preferably in a cosmetic formulation, in the form of their sodium or potassium salts when the pH of the composition is later adjusted to a pH in the range from 2.5 to 5. In the process, the free, antimicrobially active acid is released. These compositions are particularly preferred because the phosphoric esters of the present invention, unlike the carboxylic esters of the prior art, provide in this pH range a longterm-stable thickening effect which makes the use of antimicrobially active acids as preservatives possible in the first place. This makes it possible to avoid other preservatives where skin reactions are more likely, examples being halogenated preservatives.

The individual organic acids having an antimicrobial effect, preferably the carboxylic acids having an antimicrobial effect, are preferably used in the just-described compositions of the present invention in an amount of 0.05% to 2.0% by weight and more preferably in an amount of 0.1% to 1.0% by weight, based on the entire composition.

The phosphoric esters of formula (I) are also very useful as thickeners of electrolyte-containing compositions.

In a further preferred embodiment of the invention, the compositions of the present invention comprise one or more electrolytes.

The electrolytes used are inorganic salts, preferably ammonium or metal salts, more preferably of halides, for example CaCl₂, MgCl₂, LiCl, KCl and NaCl, carbonates, bicarbonates, phosphates, sulfates, nitrates, more preferably sodium chloride, and/or organic salts, preferably ammonium or metal salts, more preferably of glycolic acid, lactic acid, citric acid, tartaric acid, mandelic acid, salicylic acid, ascorbic acid, pyruvic acid, fumaric acid, retinoic acid, sulfonic acids, benzoic acid, kojic acid, fruit acid, malic acid, gluconic acid and galacturonic acid.

As electrolyte, the compositions of the present invention may also comprise mixtures of various salts.

The amount of the one or more electrolytes in the compositions of the present invention is preferably in the range from 0.1% to 20.0% by weight, more preferably in the range from 0.2% to 10.0% by weight and even more preferably in the range from 0.5% to 5.0% by weight, based on the final composition.

It is very advantageous that the phosphoric esters of formula (I) also both thicken and stabilize compositions comprising oxidizing agents, preferably hydrogen peroxide, for example hair colorants.

A further preferred embodiment of the invention are compositions of the present invention which comprise hydrogen peroxide or hydrogen peroxide releasers and which are preferably present in the form of gels or creams.

Useful hydrogen peroxide releasers preferably include inorganic peracids, preferably peroxosulfuric acid, peroxodisulfuric acid, peroxocarbonates, and also organic peracids, preferably peracetic acid.

In a further preferred embodiment of the present invention, the compositions of the present invention are acidic hydrogen peroxide bleaching gels or creams.

It is particularly in aqueous deodorant or antiperspirant formulations comprising aluminum salts, preferably aluminum chlorohydrate or aluminum-zirconium complex salts, that the phosphoric esters of formula (I) that are included therein reduce the formation of white residues on clothing donned after application of the formulations to the skin.

A further preferred embodiment of the invention are therefore compositions of the present invention characterized in that they are deodorant or antiperspirant formulations, in particular deodorant or antiperspirant formulations comprising one or more aluminum salts, preferably aluminum chlorohydrate or aluminum-zirconium complex salts.

The invention also provides for the use of one or more phosphoric esters of formula (I) in deodorant or antiperspirant formulations for reducing the formation of white residues on the clothing after using the deodorant or antiperspirant formulation on the skin.

The phosphoric esters of formula (I) are also very useful as thickener, consistency regulator, emulsifier, sensory additive, solubilizer, dispersant, glidant, adhesive and stabilizer.

The present invention therefore also provides for the use of one or more phosphoric esters of formula (I) in the compositions of the present invention as thickener, consistency regulator, emulsifier, sensory additive, solubilizer, dispersant, glidant, adhesive and stabilizer.

The use of one or more phosphoric esters of formula (I) in the compositions of the present invention as thickener is particularly preferred.

The phosphoric esters of formula (I) can advantageously be used as thickeners for compositions of the present invention on an aqueous or aqueous-alcoholic basis, for example hair gels, moisturizing gels, antiperspirant gels, bleaching gels, conditioners and disinfection gels. The phosphoric esters of formula (I) are further advantageously useful as stabilizer, dispersant and consistency regulator for aqueous-surfactant compositions of the present invention, for example shampoos, shower baths, shower gels and foam baths and for improving skin mildness and skin compatibility.

The thickening effect of the phosphoric esters of formula (I) in aqueous-surfactant compositions of the present invention is brought about by the association of the hydrophobic end groups with the surfactant micelles, and can be controlled through the choice of the ethoxylate end groups of the phosphoric esters of formula (I) and through the choice of the surfactants.

The suspending/dispersing and stabilizing effect of the phosphoric esters of formula (I) in aqueous-surfactant compositions of the present invention is due to the association of the hydrophobic end groups and of the liquid components, for example oils and silicone oils, that are insoluble in aqueous-surfactant compositions, or of the insoluble solids components, for example pigments and active ingredients such as zinc pyrethiones.

The phosphoric esters of the present invention are similarly useful as thickeners and dispersants, as emulsifiers, suspending agents having a thickening effect and consistency regulators for emulsions and suspensions, such as conditioners, and also as glidant, adhesive, thickener, dispersing and emulsifying agents of decorative, solids-containing preparations. Mixtures of the phosphoric esters of formula (I) can also be used. The emulsifying, stabilizing and/or consistency-regulating effect of the phosphoric esters of formula (I) in emulsions is caused and enhanced, respectively, by an association between the hydrophobic end groups and also by an interaction of the hydrophobic end groups with the hydrophobic oil components.

In one preferred embodiment of the present invention, the cosmetic, pharmaceutical or dermatological compositions of the present invention are present as emulsions.

The emulsions can be not only water-in-oil emulsions but also oil-in-water emulsions, microemulsions and multiple emulsions.

The emulsions can be prepared in a known manner, i.e., for example, by hot, hot/cold or PIT emulsification.

The nonaqueous portion of the emulsions, which is largely made up of the emulsifier, the thickener and the oil body, is typically in the range from 5.0% to 49.0% by weight and preferably in the range from 15.0% to 45.0% by weight. It follows that the emulsions can comprise 51.0% to 95.0% by weight and preferably 55.0% to 85.0% by weight of the aqueous phase, depending on whether lotions having a comparatively low viscosity or creams and ointments of high viscosity are to be produced.

In a further preferred embodiment of the present invention, the phosphoric esters of formula (I) are used in rinse-off products, preferably shampoos, shower baths, shower gels and foam baths.

In a further preferred embodiment of the present invention, the phosphoric esters of formula (I) are used in leave-on products, preferably skincare agents such as day creams, night creams, moisturizing lotions and gels, aqueous gels, for example facial toners, care creams, nutrient creams, body lotions, ointments, sunscreen compositions, lip care compositions, antiperspirants and deodorants.

They are further also useful for surfactant-free aqueous compositions and emulsions of the present invention and also for hair treatments, hair rinses and hair gels, but also for permanent wave compositions, hair colorants, and also for decorative cosmetics, for example make-ups, eye shadows, lipsticks, mascara and the like.

The compositions of the present invention may comprise anionic, cationic, nonionic, ampholytic surfactants and/or betaine surfactants.

The total amount of the surfactants used in the compositions of the present invention (in the case of rinse-off products for example) is, based on the final compositions of the present invention, preferably in the range from 1.0% to 48.0% by weight, more preferably in the range from 5.0% to 40.0% by weight and even more preferably in the range from 10.0% to 35.0% by weight.

The anionic surfactants are preferably (C₁₀-C₂₂)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic acid half-esters and diesters, fatty alcohol phosphates, fatty alcohol ether phosphates, protein-fatty acid condensation products, alkyl monoglyceride sulfates and sulfonates, alkyl glyceride ether sulfonates, fatty acid methyl taurides, fatty acid sarcosinates, sulforicinoleates, acyl glutamates and acyl glycinates. These compounds and mixtures thereof are used in the form of their water-soluble or water-dispersible salts for example the sodium, potassium, magnesium, ammonium, mono-, di- and triethanolammonium, and analogous alkylammonium salts.

The amount of anionic surfactants in the compositions according to the invention is preferably from 2.0 to 30.0% by weight, particularly preferably from 5.0 to 25.0% by weight and especially preferably from 12.0 to 22.0% by weight, based on the final compositions.

Preferred cationic surfactants are quaternary ammonium salts, such as di(C₈-C₂₂)-alkyldimethylammonium chloride or bromide, preferably di(C₈-C₂₂)-alkyldimethylammonium chloride or bromide; (C₈-C₂₂)-alkyldimethylethylammonium chloride or bromide; (C₈-C₂₂)-alkyltrimethylammonium chloride or bromide, preferably cetyltrimethylammonium chloride or bromide and (C₈-C₂₂)-alkyltrimethylammonium chloride or bromide; (C₁₀-C₂₄)-alkyldimethylbenzylammonium chloride or bromide, preferably (C₁₂-C₁₈)-alkyldimethylbenzylammonium chloride, (C₈-C₂₂)-alkyldimethylhydroxyethylammonium chloride, phosphate, sulfate, lactate, (C₈-C₂₂)-alkylamidopropyltrimethylammonium chloride, methosulfate, N,N-bis(2-C₈-C₂₂-alkanoyloxyethyl)dimethylammonium chloride, methosulfate, N,N-bis(2-C₈-C₂₂-alkanoyloxyethyl)hydroxyethylmethylammonium chloride, methosulfate.

The amount of cationic surfactants in the compositions according to the invention is preferably 0.1 to 10.0% by weight, particularly preferably 0.5 to 7.0% by weight and especially preferably 1.0 to 5.0% by weight, based on the final compositions.

Preferred nonionic surfactants are fatty alcohol ethoxylates (alkylpolyethylene glycols); alkylphenol polyethylene glycols; fatty amine ethoxylates (alkylaminopolyethylene glycols); fatty acid ethoxylates (acyl polyethylene glycols); polypropylene glycol ethoxylates (Pluronics®); fatty acid alkanolamides (fatty acid amide polyethylene glycols); sucrose esters; sorbitol esters and sorbitan esters and polyglycol ethers thereof, and also C₈-C₂₂-alkyl polyglucosides.

The amount of nonionic surfactants in the compositions according to the invention (e.g. in the case of rinse-off products) is preferably in the range from 1.0 to 20.0% by weight, particularly preferably from 2.0 to 10.0% by weight and especially preferably from 3.0 to 7.0% by weight, based on the final compositions.

Furthermore, the compositions according to the invention can comprise amphoteric surfactants. These can be described as derivatives of long-chain secondary or tertiary amines which have an alkyl group with 8 to 18 carbon atoms and in which a further group is substituted by an anionic group which imparts the solubility in water, thus, for example, by a carboxyl, sulfate or sulfonate group. Preferred amphoteric surfactants are N—(C₁₂-C₁₈)-alkyl-β-aminopropionates and N—(C₁₂-C₁₈)-alkyl-β-iminodipropionates as alkali metal and mono-, di- and trialkylammonium salts; suitable further surfactants are also amine oxides. These are oxides of tertiary amines with a long-chain group having 8 to 18 carbon atoms and two mostly short-chain alkyl groups having 1 to 4 carbon atoms. Preference is given here, for example, to the C₁₀- to C₁₈-alkyldimethylamine oxides, fatty acid amidoalkyldimethylamine oxide.

A further preferred group of surfactants is betaine surfactants, also called zwitterionic surfactants. These contain in the same molecule a cationic group, in particular an ammonium group and an anionic group, which may be a carboxylate group, sulfate group or sulfonate group. Suitable betaines are preferably alkylbetaines such as cocobetaine or fatty acid alkylamidopropylbetaines, for example cocoacylamidopropyldimethylbetaine or the C₁₂- to C₁₈-dimethylaminohexanoates and/or the C₁₀- to C₁₈-acylamidopropanedimethylbetaines.

The amount of amphoteric surfactants and/or betaine surfactants in the compositions according to the invention is preferably from 0.5 to 20.0% by weight and particularly preferably from 1.0 to 10.0% by weight, based on the final compositions.

Preferred surfactants are lauryl sulfate, laureth sulfate, cocoamidopropylbetaine, alkylbetaines such as cocobetaine, sodium cocoyl glutamate and lauroamphoacetate.

In a further preferred embodiment of the invention, the compositions according to the invention additionally also comprise, as foam-boosting agents, cosurfactants from the group of alkylbetaines, alkylamidobetaines, aminopropionates, aminoglycinates, imidazolinium betaines and suifobetaines, amine oxides, fatty acid alkanolamides and polyhydroxyamides.

The compositions according to the invention can comprise, as further auxiliaries and additives, oil bodies, silicone oils, waxes, emulsifiers, coemulsifiers, solubilizers, stabilizers, cationic polymers, film formers, thickeners, gelling agents, superfatting agents, refatting agents, antimicrobial active ingredients, biogenic active ingredients, astringents, deodorizing agents, sun protection filters, antioxidants, humectants, solvents, dyes, fragrances, pearlizing agents, opacifiers and/or water-soluble silicones.

The oil bodies can advantageously be selected from the groups of triglycerides, natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids or from the group of alkyl benzoates, and also natural or synthetic hydrocarbon oils.

Triglycerides of linear or branched, saturated or unsaturated, optionally hydroxylated, C₈-C₃₀-fatty acids, in particular vegetable oils, such as sunflower oil, corn oil, soybean oil, rice oil, jojoba oil, babusscu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castor oil, olive oil, peanut oil, rapeseed oil and coconut oil, and also synthetic triglyceride oils, e.g. the commercial product Myritol® 318, are suitable. Hydrogenated triglycerides are also preferred according to the present invention. Oils of animal origin, for example beef tallow, perhydrosqualene, lanolin, can also be used.

A further class of preferred oil bodies is the benzoic acid esters of linear or branched C₈₋₂₂-alkanols, e.g. the commercial products Finsoly® SB (isostearyl benzoate), Finsolv® TN (C₁₂-C₁₅-alkyl benzoate) and Finsoly® EB (ethylhexyl benzoate).

A further class of preferred oil bodies is the dialkyl ethers having in total 12 to 36 carbon atoms, in particular having 12 to 24 carbon atoms, such as, for example, di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, di-3-ethyldecyl ether, tert-butyl n-octyl ether, isopentyl n-octyl ether and 2-methylpentyl n-octyl ether, and di-tert-butyl ether and diisopentyl ether.

Branched saturated or unsaturated fatty alcohols having 6-30 carbon atoms, e.g. isostearyl alcohol, and Guerbet alcohols, are likewise suitable.

A further class of preferred oil bodies is hydroxycarboxylic acid alkyl esters. Preferred hydroxycarboxylic acid alkyl esters are full esters of glycolic acid, lactic acid, malic acid, tartaric acid or citric acid. Further esters of hydroxycarboxylic acids which are suitable in principle are esters of β-hydroxypropionic acid, of tartronic acid, of D-gluconic acid, sugar acid, mucic acid or glucuronic acid. Suitable alcohol components of these esters are primary, linear or branched aliphatic alcohols having 8 to 22 carbon atoms. Here, the esters of C₁₂-C₁₅-fatty alcohols are particularly preferred. Esters of this type are commercially available, e.g. under the trade name Cosmacol® from EniChem, Augusta lndustriale.

A further class of preferred oil bodies is dicarboxylic acid esters of linear or branched C₂-C₁₀-alkanols, such as di-n-butyl adipate (Cetiol® B), di(2-ethylhexyl)adipate and di(2-ethylhexyl) succinate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate, and also diisotridecyl azelate.

Likewise preferred oil bodies are symmetrical, asymmetrical or cyclic esters of carbonic acid with fatty alcohols, glycerol carbonate or dicaprylyl carbonate (Cetiol® CC).

A further class of preferred oil bodies is the esters of dimers of unsaturated C₁₂-C₂₂-fatty acids (dimer fatty acids) with monovalent linear, branched or cyclic C₂-C₁₈-alkanols or with polyvalent linear or branched C₂-C₆-alkanols.

A further class of preferred oil bodies is hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, and hexadecane, isohexadecane, paraffin oils, isoparaffin oils, e.g. the commercial products of the Permethyl® series, squalane, squalene, and alicyclic hydrocarbons, e.g. the commercial product 1,3-di(2-ethylhexyl)cyclohexane (Cetiol® S), ozokerite, and ceresine.

Silicone oils and silicone waxes which are available are preferably dimethylpolysiloxanes and cyclomethicones, polydialkylsiloxanes R₃SiO(R₂SiO)ₓSiR₃, where R is methyl or ethyl, particularly preferably methyl, and x is a number from 2 to 500, for example the dimethicones available under the trade names VICASIL (General Electric Company), DOW CORNING 200, DOW CORNING 225, DOW CORNING 200 (Dow Corning Corporation), and also the dimethicones available under SilCare® Silicone 41M65, SilCare® Silicone 41M70, SilCare® Silicone 41M80 (Clariant), stearyldimethylpolysiloxane, C₂₀-C₂₄-alkyldimethylpolysiloxane, C₂₄-C₂₈-alkyldimethylpolysiloxane; but also the methicones available under SilCare® Silicone 41M40, SilCare® Silicone 41M50 (Clariant), furthermore trimethylsiloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty-acid-, alcohol-, polyether-, epoxy-, fluorine- and/or alkyl-modified silicone compounds, and also polyether siloxane copolymers.

The compositions according to the invention can comprise waxes, for example paraffin waxes, microwaxes and ozokerites, beeswax and its part fractions, and also beeswax derivatives, waxes from the group of homopolymeric polyethylenes or copolymers of α-olefins, and natural waxes such as rice wax, candelilla wax, carnauba wax, Japan wax or shellac wax.

Emulsifiers, coemulsifiers and solubilizers which can be used are nonionic, anionic, cationic or amphoteric surface-active compounds.

Suitable nonionogenic surface-active compounds are preferably: addition products of from 0 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group and onto sorbitan or sorbitol esters; (C₁₂-C₁₈)-fatty acid mono- and diesters of addition products of from 0 to 30 mol of ethylene oxide onto glycerol; glycerol mono- and diesters and sorbitan mono- and diesters of saturated and unsaturated fatty acids having 6 to 22 carbon atoms and optionally ethylene oxide addition products thereof; addition products of from 15 to 60 mol of ethylene oxide onto castor oil and/or hydrogenated castor oil; polyol and in particular polyglycerol esters, such as, for example, polyglycerol polyricinoleate and polyglycerol poly-12-hydroxystearate. Ethoxylated fatty amines, fatty acid amides, fatty acid alkanolamides and mixtures of compounds of two or more of these substance classes are likewise preferably suitable.

Suitable ionogenic coemulsifiers are, for example, anionic emulsifiers, such as mono-, di- or triphosphoric acid esters, soaps (e.g. sodium stearate), fatty alcohol sulfates, but also cationic emulsifiers such as mono-, di- and trialkyl quats and polymeric derivatives thereof.

Available amphoteric emulsifiers are preferably alkylaminoalkylcarboxylic acids, betaines, sulfobetaines and imidazoline derivatives.

Fatty alcohol ethoxylates selected from the group of ethoxylated stearyl alcohols, isostearyl alcohols, cetyl alcohols, isocetyl alcohols, oleyl alcohols, lauryl alcohols, isolauryl alcohols, cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether are particularly preferably used.

Fatty acid ethoxylates selected from the group of ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20)oleate are likewise preferred.

Sodium laureth-11 carboxylate can advantageously be used as ethoxylated alkylether carboxylic acid or salts thereof.

Ethoxylated triglycerides which can be used are advantageously polyethylene glycol(60) evening primrose glycerides.

It is furthermore advantageous to select the polyethylene glycol glycerol fatty acid esters from the group polyethylene glycol(20) glyceryl laurate, polyethylene glycol(6) glyceryl caprate/caprinate, polyethylene glycol(20) glyceryl oleate, polyethylene glycol(20) glyceryl isostearate and polyethylene glycol(18) glyceryl oleate/cocoate.

Among the sorbitan esters, polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate are particularly suitable.

Particularly advantageous coemulsifiers are glyceryl monostearate, glyceryl monooleate, diglyceryl monostearate, glyceryl isostearate, polyglyceryl-3 oleate, polyglyceryl-3 diisostearate, polyglyceryl-4 isostearate, polyglyceryl-2 dipolyhydroxystearate, polyglyceryl-4 dipolyhydroxystearate, PEG-30 dipolyhydroxystearate, diisostearoyl polyglyceryl-3 diisostearate, glycol distearate and polyglyceryl-3 dipolyhydroxystearate, sorbitan monoisostearate, sorbitan stearate, sorbitan oleate, sucrose distearate, lecithin, PEG-7-hydrogenated castor oil, cetyl alcohol, stearyl alcohol, behenyl alcohol, isobehenyl alcohol and polyethylene glycol(2) stearyl ether (steareth-2), alkylmethicone copolyols and alkyldimethicone copolyols, in particular cetyldimethicone copolyol, laurylmethicone copolyol.

The compositions according to the invention can comprise one or more of the emulsifiers, coemulsifiers or solubilizers in amounts of from 0.1 to 20.0% by weight, preferably from 1.0 to 15.0% by weight and particularly preferably from 3.0 to 10.0% by weight, based on the final compositions.

Stabilizers which can be used are metal salts of fatty acids, such as, for example, magnesium stearate, aluminum stearate and/or zinc stearate, preferably in amounts of from 0.1 to 10.0% by weight, preferably from 0.5 to 8.0% by weight and particularly preferably from 1.0 to 5.0% by weight, based on the final compositions.

Suitable cationic polymers are those known under the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and Polyquaternium 37&mineral oil&PPG trideceth (Salcare SC95), PVP-dimethylaminoethyl methacrylate copolymer, guar hydroxypropyltriammonium chlorides, and calcium alginate and ammonium alginate. Furthermore, cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicones; copolymers of adipic acid and dimethylaminohydroxypropyldiethylenetriamine; polyaminopolyamide and cationic chitin derivatives, such as, for example, chitosan, can be used.

The compositions according to the invention can comprise one or more of the aforementioned cationic polymers in amounts of from 0.1 to 5.0% by weight, preferably from 0.2 to 3.0% by weight and particularly preferably from 0.5 to 2.0% by weight, based on the final compositions.

Furthermore, the compositions according to the invention can comprise film formers which, depending on the intended use, are selected from salts of phenylbenzimidazolesulfonic acid, water-soluble polyurethanes, for example C₁₀-polycarbamyl polyglyceryl ester, polyvinyl alcohol, polyvinylpyrrolidone copolymers, for example vinylpyrrolidone/vinyl acetate copolymer, water-soluble acrylic acid polymers/copolymers and esters or salts thereof, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate, for example available under the trade name Aristoflex® A 60 (Clariant).

The compositions according to the invention can comprise one or more film formers in amounts of from 0.1 to 10.0% by weight, preferably from 0.2 to 5.0% by weight and particularly preferably from 0.5 to 3.0% by weight, based on the final compositions.

The desired viscosity of the compositions can be established by adding further thickeners and gelling agents. Of suitability are preferably cellulose ethers and other cellulose derivatives (e.g. carboxymethylcellulose, hydroxyethylcellulose), gelatin, starch and starch derivatives, sodium alginates, fatty acid polyethylene glycol esters, agar, tragacanth or dextrin derivatives, in particular dextrin esters. Furthermore, metal salts of fatty acids, preferably having 12 to 22 carbon atoms, for example sodium stearate, sodium palmitate, sodium laurate, sodium arachidates, sodium behenate, potassium stearate, potassium palmitate, sodium myristate, aluminum monostearate, hydroxy fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures of such are suitable. Furthermore, crosslinked and uncrosslinked polyacrylates such as carbomers, sodium polyacrylates or polymers containing sulfonic acid, such as ammonium acryloyldimethyltaurate/VP copolymer, can be used.

Preferably, the compositions according to the invention comprise 0.01 to 20.0% by weight, particularly preferably 0.1 to 10.0% by weight, especially preferably 0.2 to 3.0% by weight and extraordinarily preferably 0.4 to 2.0% by weight, of thickeners and/or gelling agents.

Superfatting agents which can be used are preferably lanolin and lecithin, nonethoxylated and polyethoxylated or acylated lanolin derivatives and lecithin derivatives, polyol fatty acid esters, mono-, di- and triglycerides and/or fatty acid alkanolamides, where the latter simultaneously serve as foam stabilizers, which are preferably used in amounts of from 0.01 to 10.0% by weight, particularly preferably from 0.1 to 5.0% by weight and especially preferably from 0.5 to 3.0% by weight, based on the final compositions according to the invention.

The antimicrobial active ingredients used are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether(triclosan), phenoxyethanol, 1,5-pentanediol, 1,6-hexanediol, 3,4,4′-trichlorocarbanilide(triclocarban), diaminoalkylamide, for example L-lysine hexadecylamide, citrate heavy metal salts, salicylates, piroctoses, in particular zinc salts, pyrithiones and heavy metal salts thereof, in particular zinc pyrithione, zinc phenol sulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and Octopirox®, iodopropynyl butylcarbamate, methylchioroisothiazolinone, methylisothiazolinone, methyldibromoglutaronitrile, AgCl, chloroxylenol, Na salt of diethylhexyl sulfosuccinate, sodium benzoate, and phenoxyethanol, benzyl alcohol, phenoxyisopropanol, parabens, preferably butyl, ethyl, methyl and propyl paraben, and Na salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), Na salt of hydroxymethylglycinate, hydroxyethylglycine of sorbic acid and combinations of these active substances.

The compositions according to the invention comprise the antimicrobial active ingredients preferably in amounts of from 0.001 to 5.0% by weight, particularly preferably from 0.01 to 3.0% by weight and especially preferably from 0.1 to 2.0% by weight, based on the final compositions.

The compositions according to the invention can furthermore comprise biogenic active ingredients selected from plant extracts, such as, for example, aloe vera, and also local anesthetics, antibiotics, antiphlogistics, antiallergics, corticosteroids, sebostatics, Bisabolol®, allantoin, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

The compositions according to the invention can comprise biogenic active ingredients preferably in amounts of from 0.001 to 5.0% by weight, particularly preferably from 0.01 to 3.0% by weight and especially preferably from 0.1 to 2.0% by weight, based on the final compositions.

The compositions according to the invention can comprise astringents, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc, and also aluminum chlorohydrates, preferably in amounts of from 0 to 50.0% by weight, particularly preferably in amounts of from 0.01 to 10.0% by weight and especially preferably in amounts of from 01 to 10.0% by weight. Allantoin and bisabolol are preferred as deodorizing substances. These are preferably used in amounts of from 0.0001 to 10.0% by weight.

The compositions according to the invention can comprise microfine titanium dioxide, mica-titanium oxide, iron oxides, mica-iron oxide, zinc oxide, silicon oxides, ultramarine blue, chromium oxides as pigments/micropigments and also as sun protection filters.

The compositions according to the invention can comprise sun protection filters, preferably selected from 4-aminobenzoic acid, 3-(4′-trimethylammonium)benzylideneboran-2-one methyl sulfate, camphorbenzalkoniummethosulfate, 3,3,5-trimethylcyciohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethine)bis(7,7-dimethyl-2-oxobicyclo[2.2.1]heptane-1-methanesulfonic acid) and its salts, 1-(4-tert-butylphenyl)-3-(4-methoxyphenyl)propane-1,3-dione, 3-(4′-sulfo)benzylidenebornan-2-one and its salts, 2-ethylhexyl 2-cyano-3,3-diphenylacrylate, polymers of N-[2(and 4)-(2-oxoborn-3-ylidenemethyl)benzyl]acrylamide, 2-ethylhexyl 4-methoxycinnamate, ethoxylated ethyl 4-aminobenzoate, isoamyl 4-methoxycinnamate, 2,4,6-tris[p-(2-ethylhexyloxycarbonyl)anilino]-1,3,5-triazine, 2-(2H-benzotriazol-2-yl)-4-methyl-6-(2-methyl-3-(1,3,3,3-tetramethyl-1-(trimethylsilyloxy)disiloxanyl)propyl)phenol, bis(2-ethylhexyl) 4,4′-[(6-[4-((1,1-dimethylethyl)aminocarbonyl)phenylamino}-1,3,5-triazin-2,4-yl)diimino]bisbenzoate, benzophenone-3, benzophenone-4 (acid), 3-(4′-methylbenzylidene)-D,L-camphor, 3-benzylidenecamphor, 2-ethylhexyl salicylate, 2-ethylhexyl 4-dimethylaminobenzoate, hydroxy-4-methoxybenzophenone-5-sulfonic acid (sulfisobenzone) and the sodium salt, 4-isopropylbenzyl salicylate, N,N,N-trimethyl-4-(2-oxoborn-3-ylidenemethyl)anilium methyl sulfate, homosalate (INN), oxybenzone (INN), 2-phenylbenzimidazole-5-sulfonic acid and its sodium, potassium and triethanolamine salts, octylmethoxycinnamic acid, isopentyl-4-methoxycinnamic acid, isoamyl-p-methoxycinnamic acid, 2,4,6-trianilino(p-carbo-2′-ethylhexyl-1′-oxy)-1,3,5-triazine (octyltriazone) phenol, 2-2(2H-benzotriazol-2-yl)-4-methyl-6-(2-methyl-3-(1,3,3,3-tetramethyl-1-(trimethylsilyl)oxy)disiloxanyl)propyl(drometrizoletrisiloxane)benzoic acid, 4,4-((6-(((1,1-dimethylethyl)amino)carbonyl)phenyl)amino)-1,3,5-triazine-2,4-diyl)diimino)bis,bis(2-ethylhexyl) ester)benzoic acid, 4,4-((6-(((1,1-dimethylethyl)amino)carbonyl)phenyl)amino)-1,3,5-triazine-2,4-diyl)diimino)bis,bis(2-ethylhexyl)ester), 3-(4′-methylbenzylidene)-D,L-camphor (4-methylbenzylidenecamphor), benzylidenecamphorsulfonic acid, octocrylene, polyacrylamidomethylbenzylidenecamphor, 2-ethylhexyl salicylate (octylsalicylate), ethyl-2-hexyl 4-dimethylaminobenzoate (octyldimethyl PABA), PEG-25 PABA, 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate, di-p-methoxycinnamic acid, p-aminobenzoic acid and esters thereof, 4-tert-butyl-4′-methoxydibenzoylmethane, 4-(2-β-glucopyranoxy)propoxy-2-hydroxybenzophenone, octyl salicylate, methyl-2,5-diisopropylcinnamic acid, cinoxate, dihydroxydimethoxybenzophenone, disodium salt of 2,2′-dihydroxy-4,4′-dimethoxy-5,5′-disulfobenzophenone, dihydroxybenzophenone, 1,3,4-dimethoxyphenyl-4,4-dimethyl-1,3-pentanedione, 2-ethylhexyl dimethoxybenzylidenedioxoimidazolidinepropionate, methylenebisbenzotriazolyl tetramethylbutyiphenol, phenyl dibenzimidazoletetrasulfonate, bis-ethylhexyloxyphenol methoxyphenol triazine, tetrahydroxybenzophenones, terephthalylidenedicamphorsulfonic acid, 2,4,6-tris[4,2-ethylhexyloxycarbonyl)anilino}-1,3,5-triazine, methylbis(trimethylsiloxy)silylisopentyltrimethoxycinnamic acid, amyl p-dimethylaminobenzoate, amyl p-dimethylaminobenzoate, 2-ethylhexyl p-dimethylaminobenzoate, isopropyl-p-methoxycinnamic acid/diisopropylcinnamic acid esters, 2-ethylhexyl-p-methoxycinnamic acid, 2-hydroxy-4-methoxybenzophenone, 2-hydroxy-4-methoxybenzophenone-5-sulfonic acid and the trihydrate, and also 2-hydroxy-4-methoxybenzophenone-5-sulfonate sodium salt and phenylbenzimidazolesulfonic acid.

The amount of the aforementioned sun protection filters (one or more compounds) in the compositions of the present invention is preferably 0.001 to 30.0% by weight, particularly preferably 0.05 to 20.0% by weight and especially 1.0 to 10.0% by weight, based on the total weight of the final composition.

The compositions according to the invention can comprise antioxidants, preferably selected from amino acids (e.g. glycine, histidine, tyrosine, tryptophan) and derivatives thereof, imidazoles (e.g. urocanic acid) and derivatives thereof, peptides such as DL-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g, thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and also salts thereof, dilauryl thiodipropionate, distearyl thiodipropionate, thiodipropionic acid and derivatives thereof (e.g. esters, ethers, peptides, lipids, nucleotides, nucleosides and salts), and also sulfoximine compounds (e.g. buthionine sulfoximines, homocysteine sulfoximine, buthionine sulfones, penta-, hexa-, heptathionine sulfoximine) in very low tolerated doses, also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide), superoxide dismutase and the derivatives suitable according to the invention (salts, esters, ethers, sugars, nucleotides, nucleosides, peptides and lipids) of these specified substances.

The antioxidants can protect the skin and the hair against oxidative stress. Preferred antioxidants here are vitamin E and derivatives thereof, and vitamin A and derivatives thereof.

The amount of the one or more antioxidants in the compositions according to the invention is preferably 0.001 to 30.0% by weight, particularly preferably 0.05 to 20.0% by weight and especially preferably 1.0 to 10.0% by weight, based on the total weight of the composition.

Furthermore, humectants selected from the sodium salt of 2-pyrrolidone-5-carboxylate (NaPCA), guanidine; glycolic acid and salts thereof, lactic acid and salts thereof, glucosamines and salts thereof, lactamide monoethanolamine, acetamide monoethanolamine, urea, hydroxy acids, panthenol and derivatives thereof, for example D-panthenol (R-2,4-dihydroxy-N-(3-hydroxypropyl)-3,3-dimethylbutamide), DL-panthenol, calcium pantothenate, panthetine, pantotheine, panther yl ethyl ether, isopropyl palmitate, glycerol and/or sorbitol can be used, preferably in amounts of from 0.1 to 15.0% by weight and particularly preferably from 0.5 to 5.0% by weight, based on the final compositions.

Additionally, the compositions according to the invention can comprise organic solvents. In principle, suitable organic solvents are all mono- or polyhydric alcohols. Preference is given to using alcohols having 1 to 4 carbon atoms, such as ethanol, propanol, isopropanol, n-butanol, isobutanol, t-butanol, glycerol and mixtures of said alcohols. Further preferred alcohols are polyethylene glycols with a relative molecular mass below 2000. In particular, a use of polyethylene glycol with a relative molecular mass between 200 and 600 and in amounts up to 45.0% by weight and of polyethylene glycol with a relative molecular mass between 400 and 600 in amounts of from 5.0 to 25.0% by weight is preferred. Further suitable solvents are, for example, triacetin (glycerol triacetate) and 1-methoxy-2-propanol.

The dyes and color pigments present in the compositions according to the invention, both organic and inorganic dyes, are selected from the corresponding positive list of the Cosmetics Regulations or the EU list of cosmetic colorants.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Cerise Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfonic acid diethylamide-1′-phenylazo)-3- | 12490 | red |
| hydroxy-5″-chloro-2″,4″-dimethoxy-2-naphthoic acid anilide | | |
| Disperse Yellow 16 | 12700 | yellow |
| 1-(4-Sulfo-1-phenylazo)-4-aminobenzenesulfonic acid | 13015 | yellow |
| 2,4-Dihydroxyazobenzene-4′-sulfonic acid | 14270 | orange |
| 2-(2,4-Dimethylphenylazo-5-sulfonic acid)-1-hydroxy- | 14700 | red |
| naphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonic acid-4-chloro-5-carboxylic acid-1-phenylazo)- | 15525 | red |
| 2-hydroxynaphthalene | | |
| 1-(3-Methylphenylazo-4-sulfonic acid)-2-hydroxynaphthalene | 15580 | red |
| 1-(4′,(8′)-Sulfonic acid naphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2-hydroxy- | 15865 | red |
| naphthalene-3-carboxylic acid | | |
| 1-(2-Sulfo-1-naphthylazo)-2-hydroxynaphthalene- | 15880 | red |
| 3-carboxylic acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| pyrazolone-3-carboxylic acid | | |
| Pigment Yellow 16 | 20040 | yellow |
| 2,6-((4′-Sulfo-2″,4″-dimethyl)bisphenylazo)-1,3-dihydroxy- | 20170 | orange |
| benzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]- | 27755 | black |
| 1-hydroxy-7-aminonaphthalene-3,6-disulfonic acid | | |
| 4′-[(4″-Sulfo-1″-phenylazo)-7′-sulfo-1′-naphthylazo]- | 28440 | black |
| 1-hydroxy-8-acetylaminonaphthalene-3,5-disulfonic acid | | |
| Direct Orange 34, 39, 44, 46, 60 | 40215 | orange |
| Food Yellow | 40800 | orange |
| trans-β-Apo-8′-Carotenealdehyde (C₃₀) | 40820 | orange |
| trans-Apo-8′-Carotenic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethylamino)triphenyl- | 42051 | blue |
| carbinol | | |
| 4-[(4-N-Ethyl-p-sulfobenzylamino)phenyl(4-hydroxy- | 42053 | green |
| 2-sulfophenyl)(methylene)-1-(N-ethyl-N-p-sulfobenzyl)- | | |
| 2,5-cyclohexadieneimine] | | |
| Acid Blue 7 | 42080 | blue |
| (N-Ethyl-p-sulfobenzylaminophenyl(2-sulfophenyl)- | 42090 | blue |
| methylene(N-ethyl-N-p-sulfobenzyl)cyclohexadieneimine | | |
| Acid Green 9 | 42100 | green |
| Diethyldisulfobenzyldi-4-amino-2-chlorodi-2-methyl- | 42170 | green |
| fuchsonimmonium | | |
| Basic Violet 14 | 42510 | violet |
| Basic Violet 2 | 42520 | violet |
| 2′-Methyl-4′-(N-ethyl-N-m-sulfobenzyl)amino-4″-(N-diethyl)- | 42735 | blue |
| amino-2-methyl-N-ethyl-N-m-sulfobenzylfuchsonimmonium | | |
| 4′-(N-Dimethyl)amino-4″-(N-phenyl)aminonaphtho- | 44045 | blue |
| N-dimethylfuchsonimmonium | | |
| 2-Hydroxy-3,6-disulfo-4,4′-bisdimethylaminonaphtho- | 44090 | green |
| fuchsinimmonium | | |
| Acid red | 45100 | red |
| 3-(2′-Methylphenylamino)-6-(2′-methyl-4′-sulfophenylamino)- | 45190 | violet |
| 9-(2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidine)anthraquinone | 61570 | green |
| Acid Blue 80 | 61585 | blue |
| Acid Blue 62 | 62045 | blue |
| N,N′-Dihydro-1,2,1′,2′-anthraquinoneazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6, 19; Natural Red 1 | 75100 | yellow |
| Bixin, Nor-Bixin | 75120 | orange |
| Lycopene | 75125 | yellow |
| trans-alpha, beta- or gamma-Carotene | 75130 | orange |
| Keto- and/or hydroxyl derivatives of carotene | 75135 | yellow |
| Guanine or pearlescent agents | 75170 | white |
| 1,7-Bis(4-hydroxy-3-methoxyphenyl)-1,6-heptadiene- | 75300 | yellow |
| 3,5-dione | | |
| Complex salt (Na, Al, Ca) of carminic acid | 75470 | red |
| Chlorophyll a and b; copper compounds of the chlorophylls | 75810 | green |
| and chlorophyllines | | |
| Aluminum | 77000 | white |
| Aluminum hydrate | 77002 | white |
| Water-containing aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromic oxide | 77288 | green |
| Chromic oxide, water-containing | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxides and hydroxides | 77491 | red |
| Hydrated iron oxide | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂•7H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, lactoflavin | | yellow |
| Caramel | | brown |
| Capsanthin, Capsorubin | | orange |
| Betanine | | red |
| Benzopyrilium salts, anthocyanines | | red |
| Aluminum, zinc, magnesium and calcium stearate | | white |
| Bromothymol Blue | | blue |
| Bromocresol Green | | green |
| Acid Red 195 | | red |

Oil-soluble natural dyes, such as, for example, paprika extracts, f3-carotene and cochineal are furthermore advantageous.

Also advantageously used are pearlescent pigments, e.g. pearl essence (guanine/hypoxanthine mixed crystals from fish scales) and mother of pearl (ground mussel shells), monocrystalline pearlescent pigments such as, for example, bismuth oxychloride (BiOCl), layer substrate pigments, e.g. mica/metal oxide, silver-white pearlescent pigments from TiO₂, interference pigments (TiO₂, variable layer thickness), color luster pigments (Fe₂O₃) and combination pigments (TiO₂/Fe₂O₃, TiO₂/Cr₂O₃, TiO₂/Prussian blue, TiO₂/carmine).

Effect pigments within the context of the present invention are understood as meaning pigments which due to their refraction properties produce special optical effects. Effect pigments impart to the treated surface (skin, hair, mucous membrane) luster or glitter effects or can visually conceal unevenness of the skin and skin wrinkles by means of diffuse light scattering. As a particular embodiment of the effect pigments, interference pigments are preferred. Particularly suitable effect pigments are, for example, mica particles which are coated with at least one metal oxide. Besides mica, a sheet silicate, silica gel and other SiO₂ modifications are also suitable as carriers. A metal oxide frequently used for coating is, for example, titanium oxide, to which, if desired, iron oxide can be admixed. By means of the size and shape (e.g. spherical, ellipsoidal, flat, even, uneven) of the pigment particles and by means of the thickness of the oxide coating, the reflection properties can be influenced. Other metal oxides, e.g. bismuth oxychloride (BiOCI), and the oxides of, for example, titanium, in particular the TiO₂ modifications anatase and rutile, aluminum, tantalum, niobium, zirconium and hafnium can also be used. Effect pigments can also be prepared using magnesium fluoride (Mg F₂) and calcium fluoride (fluorspar, CaF₂).

The effects can be controlled both by means of the particle size and by means of the particle size distribution of the pigment ensemble. Suitable particle size distributions extend, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A wider particle size distribution, for example of 20-150 μm, produces glittering effects, whereas a narrower particle size distribution of <15 μm provides for a uniform silky appearance.

The compositions of the present invention comprise effect pigments preferably in amounts from 0.1% to 20.0% by weight, more preferably from 0.5% to 10.0% by weight and even more preferably from 1.0% to 5.0% by weight, all based on the total weight of the composition.

Preference as deodorizing substances is given to allantoin and bisabolol. These are preferably used in amounts from 0.0001% to 10.0% by weight.

Fragrance and/or perfume oils which may be used are individual odorant compounds, e.g. the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethol, citronellol, eugenol, geraniol, linalool, phenylethyl alcohol and terpineol; and the hydrocarbons include primarily the terpenes and balsams. Preference is given to using mixtures of different odorants which together produce a pleasing scent note.

Perfume oils can also comprise natural odorant mixtures, as are accessible from vegetable or animal sources, e.g. pine oil, citrus oil, jasmine oil, lily oil, rose oil or ylang-ylang oil. Essential oils of relatively low volatility, which in most cases are used as aromatic components, are also suitable as perfume oils, e.g. sage oil, chamomile oil, clove oil, melissa oil, mint oil, cinnamon leaf oil, linden blossom oil, juniperberry oil, vetiver oil, olibanum oil, galbanum oil and ladanum oil.

Preferably suitable as pearlizing component are fatty acid monoalkanolamides, fatty acid dialkanolamides, monoesters or diesters of alkylene glycols, in particular ethylene glycol and/or propylene glycol or oligomers thereof, with higher fatty acids, such as, for example, palmitic acid, stearic acid and behenic acid, monoesters or polyesters of glycerol with carboxylic acids, fatty acids and metal salts thereof, ketosulfones or mixtures of the specified compounds. Particular preference is given to ethylene glycol distearates and/or polyethylene glycol distearates having on average 3 glycol units.

When the compositions according to the invention comprise pearlizing compounds, these are preferably present in the compositions according to the invention in an amount of from 0.1 to 15.0% by weight and particularly preferably in an amount of from 1.0 to 10.0% by weight.

The acids or alkalis used for adjusting the pH are preferably mineral acids, in particular HCl, inorganic bases, in particular NaOH or KOH, and organic acids, in particular citric acid.

The following examples and applications are intended to further elucidate the invention without, however, limiting it thereto. All percentages are percent (%) by weight.

### PREPARATION EXAMPLES, GENERAL OPERATING PROCEDURE

In the preparation of the phosphoric esters of formula (I), phosphoric acid (85% strength), and fatty alcohol ethoxylate are used in a certain molar ratio. For this purpose, all starting materials are initially charged in a stirred apparatus equipped with heating mantle, separator with condenser and vacuum connection. The mixture is heated to 100° C. and three times evacuated down to 100 mbar and subsequently refilled with nitrogen. After a further 4 hours of inertization (nitrogen being introduced at 20 liters/hour) at 100° C., the batch is heated to 230° C. while nitrogen is being introduced and esterified (water removed in the separator). The reaction times are 24 to 42 hours (reckoned from 230° C. esterification temperature), particularly 40 hours. The .residual acid number is then <3 mg KOH/g. This corresponds approximately to 93 to 96% conversion (based on starting acid number). After the reaction has ended, the product is cooled to 80° C. and poured into a dish and the solidified melt is comminuted.

### Example 1

Ester from 12.7 g of phosphoric acid and 701.3 g of ceteareth-50 (C_{16/18} fatty alcohol+50 mol of ethylene oxide) in a molar ratio of 1:3 residual acid number: 0.8 mg KOH/g (97% conversion), ³¹P NMR: diester/triester=13/87 mol %.

### Example 2

Ester from 11.4 g of phosphoric acid and 935.1 g of ceteareth-80 (C_{16/18} fatty alcohol+80 mol of ethylene oxide) in a molar ratio of 1:3 residual acid number: 0.8 mg KOH/g (96% conversion), ³¹P NMR: diester/triester=15/85 mol %.

### Comparative Example 1

Ester from 17.3 g of phosphoric acid and 666.0 g of ceteareth-25 (C_{16/18} fatty alcohol+25 mol of ethylene oxide) in a molar ratio of 1:3 residual acid number: 1.7 mg KOH/g (95% conversion), ³¹P NMR: diester/triester=11/89 mol %.

Viscosity Measurements in Aqueous Compositions

The viscosities of the phosphoric esters of Examples 1 and 2 and also of Comparative Example 1 compared with the commercial products Crothix (PEG-150 Pentaerythrityl Tetrastearate), Genapol® DAT 100 (PEG-150 Polyglyceryl-2 Tristearate) and Rewopal® PEG 6000 DS (PEG-150 Distearate) were measured (in each case 6% by weight of product in water). The viscosity was measured at 20° C. with an RVT type Brookfield viscometer at 20 rpm immediately after preparation of the aqueous compositions (“viscosity as-prepared”) and after 3 months' storage at room temperature (RT).

TABLE 1
Viscosities of aqueous compositions

| | Viscosity | | Viscosity after 3 months' |
|---|---|---|---|
| | as-prepared | | storage at RT |
| Product | [mPa · s] | Clarity | [mPa · s] |
| Example 1 | 29400 | clear | 23000 |
| Example 2 | 19200 | clear | 16000 |
| Comparative | 58 | clear | 40 |
| Example 1 | | | |
| PEG-150 | 6650 | cloudy | 990 |
| Pentaerythrityl | | | |
| tetrastearate | | | |
| PEG-150 | 65 | cloudy | n.d. |
| Polyglyceryl | | | |
| 2-tristearate | | | |
| PEG-150 Distearate | 3900 | cloudy | n.d. |

RT: room temperature; n.d. not determined

The results in table 1 show that Inventive Examples 1 and 2 form clear high-viscosity gels in water, which retain their viscosity even after 3 months' storage at room temperature (RT). Phosphoric esters having a lower degree of ethoxylation (derived from C_{16/18} fatty alcohol+25 mol of ethylene oxide) display an only very weakly developed thickening performance.

Viscosity Measurements in Aqueous-Surfactant Compositions

The viscosities of the phosphoric esters of Examples 1 and 2 and of Comparative Example 1 compared with the commercial products Crothix (PEG-150 Pentaerythrityl Tetrastearate), Genapol® DAT 100 (PEG-150 Polyglyceryl-2 Tristearate) and Rewopal® PEG 6000 DS (PEG-150 Distearate) were measured (in each case 1% by weight of product in an aqueous solution of sodium Iauryl ether sulfate: cocoamidopropylbetaine in a ratio of 8:2 having a total surfactant content of 15% by weight in water; pH 4-4.4). The viscosity was measured at 20° C. with an RVT type Brookfield viscometer at 20 rpm immediately after preparation of the aqueous-surfactant compositions (“viscosity as-prepared”) and after 3 months' storage at 50° C.

TABLE 2
Viscosities of aqueous-surfactant compositions

| | Viscosity as- | Viscosity after 3 months' |
|---|---|---|
| | prepared | storage at 50° C. |
| Product | [mPa · s] | [mPa · s] |
| Example 1 | 61000 | 63000 |
| Example 2 | 24700 | 30000 |
| Comparative Example 1 | 785 | 400 |
| PEG-150 Pentaerythrityl | 20000 | 80 |
| tetrastearate | | |
| PEG-150 | 23100 | 110 |
| Polyglyceryl-2-tristearate | | |
| PEG-150 Distearate | 2750 | 45 |

The results in table 2 show that Inventive Examples 1 and 2 form high-viscosity gels in a surfactant-containing formulation that retain their viscosity even after 3 months' storage at 50° C.

The formulation examples which follow demonstrate the use of the phosphoric esters of formula (I) in inventive aqueous and surfactant formulations.

### Formulation Example 1

### Facial Cleansing Foam

| | | |
|---|---|---|
| A | stearic acid | 1.60% |
| | myristic acid | 1.80% |
| | lauric acid | 0.70% |
| | Tegin M | 0.50% |
| | Glyceryl Stearate | |
| | palmitic acid | 0.70% |
| B | water | ad 100.00% |
| C | potassium hydroxide | 0.70% |
| | phosphoric ester of Example 1 | 1.00% |

Preparation:
I Melt A at 80° C.
II Dissolve C in B with stirring and at 60° C., then add to I.
III Cool down with stirring.

Formulation Example 2

### Cream Rinse

| | | | |
|---|---|---|---|
| A | Genamin ® CTAC | (Clariant) | 6.00% |
| | Cetrimonium Chloride | | |
| | Hostacerin ® DGL | (Clariant) | 1.50% |
| | PEG-10 Diglyceryl-2 Laurate | | |
| | Cetylstearyl Alcohol | | 1.70% |
| | paraffin oil | | 1.00% |
| B | water | | ad 100.00% |
| C | phosphoric ester of Example 2 | | 1.00% |
| D | perfume | | 0.30% |
| | panthenol | | 0.30% |
| | preservative | | q.s. |
| | dye | | q.s. |

Preparation:
I Dissolve A at 75° C.
II Dissolve C in B with stirring at 60° C.
III Add II to I with stirring. Stir until cold.
IV At 40° C. add the components of D.
V Adjust the pH to 4.

### Formulation Example 3

### Light Leave on for Hair Tips

| A | SilCare ® Silicone 41M15 | (Clariant) | 0.30% |
|---|---|---|---|
| | Caprylyl Methicone | | |
| B | Genapol ® LA 070 | (Clariant) | 8.00% |
| | Laureth-7 | | |
| C | water | | ad 100% |
| D | phosphoric ester of Example 2 | | 2.00% |
| E | Biobranil | | 0.50% |
| | Soybean (Glycine Soja) Oil and Wheat | | |
| | (Triticum Vulgare) Bran Lipids | | |
| | glycerol | | 2.00% |
| | panthenol | | 0.50% |
| F | SilCare ® Silicone SEA | (Clariant) | 0.50% |
| | Trideceth-9 PG Amodimethicone and Trideceth-12 | | |
| | Genamin ® CTAC | (Clariant) | 2.00% |
| | Cetrimonium Chloride | | |
| | Nipaguard ® DMDMH | (Clariant) | 0.20% |
| | DMDMH Hydantoin | | |

Preparation:
I Solubilize A in B.
II Dissolve D in C with stirring at 60° C.
III Add E to II and stir until the solution is clear, then add to I.
IV Add F to III.

Formulation Example 4

### Hydrogen Peroxide Gel

| | | | |
|---|---|---|---|
| A | phosphoric ester of Example 1 | | 5.00% |
| | Genapol ® T 250 | (Clariant) | 2.00% |
| | Ceteareth-25 | | |
| B | water | | ad 100.00% |
| C | phosphoric acid | | 0.04% |
| | sodium dihydrogen phosphate | | 1.00% |
| D | hydrogen peroxide 30% strength | | 18.00% |

Preparation:
I Dissolve A in B with stirring and heating to 50° C.
II Add C at 25° C.
III Add D at room temperature.

### Formulation Example 5

### Deodorant Gel

| A | Octopirox ® | (Clariant) | 0.10% |
|---|---|---|---|
| | Piroctone Olamine | | |
| B | Emulsogen ® HCP 049 | (Clariant) | 10.00% |
| | PEG-40 Hydrogenated Castor Oil and Propylene Glycol | | |
| | perfume | | 0.20% |
| C | water | | ad 100.00% |
| D | phosphoric ester of Example 2 | | 3.00% |
| E | citric acid | | q.s. |

Preparation:
I Dissolve A in B.
II Dissolve D in C with stirring and slight heating, then add II to I.
III If necessary, adjust the pH to 6.0 with E.

### Formulation Example 6

### Make-Up Remover

| | | | |
|---|---|---|---|
| A | Velsan ® P8-3 | (Clariant) | 5.00% |
| | Isopropyl C12-15 Pareth-9 Carboxylate | | |
| B | Hostapon ® CGN | (Clariant) | 2.00% |
| | Sodium Cocoyl Glutamate | | |
| | Genagen ® CAB | (Clariant) | 3.00% |
| | Cocamidopropyl Betaine | | |
| | Allantoin | (Clariant) | 0.30% |
| | Aristoflex ® PEA | (Clariant) | 1.00% |
| | Polypropylene Terephthalate | | |
| | 1.6 Hexanediol | | 2.00% |
| | 1.2 Propanediol | | 2.00% |
| | Polyglycol 400 | (Clariant) | 2.00% |
| | PEG-8 | | |
| | panthenol | | 0.50% |
| | Lutrol F 127 | | 3.00% |
| | Poloxamer 407 | | |
| | preservative | | q.s. |
| C | phosphoric ester of Example 1 | | 1.00% |
| D | water | | ad 100.00% |
| E | Genapol ® LA 070 | (Clariant) | 2.00% |
| | Laureth-7 | | |

Preparation:
I A little at a time add the components of B to A and stir until a clear solution forms.
II Dissolve C in D with stirring and slight heating, add II to I.
III Stir E into I.

### Formulation Example 7

### Whitening Gel

| A | water | ad 100.00% |
|---|---|---|
| | arginine | 1.10% |
| | phosphoric ester of Example 1 | 4.00% |
| B | dipropylene glycol | 8.00% |
| | Genapol ® C 100 (Clariant) | 0.60% |
| | Coceth-10 | |
| | Sodium citrate*2H₂O | 0.09% |
| | citric acid 10.0% | 0.10% |
| | Nipagin ® M (Clariant) | 0.20% |
| | Methylparaben ascorbic acid 2-glucoside | 2.00% |

Preparation:
I Mix the components of A and dissolve with stirring and slight heating.
II Add the components of B to I and dissolve. If necessary, heat the formulation slightly.

### Formulation Example 8

### Facial Toner

| | | |
|---|---|---|
| A | glycerol | 8.00% |
| | Polyglycol 400 (Clariant) | 5.00% |
| | PEG-8 | |
| | panthenol | 0.50% |
| | perfume | 0.20% |
| | alcohol | 8.00% |
| | preservative | q.s. |
| | Allantoin (Clariant) | 0.10% |
| | Niacinamide | 0.10% |
| | Extrapon Hamamelis | 1.00% |
| | water, Witch Hazel Distillate, SD Alcohol 39-C, | |
| | Butylene Glycols | |
| B | water | ad 100% |
| C | phosphoric ester of Example 2 | 2.00% |

Preparation:
I Dissolve C in B with stirring and slight heating.
II Add the components of A to I and stir until formulation is homogeneous.

### Formulation Example 9

### Hair Shampoo

| | | |
|---|---|---|
| A | Genapol ® LRO liquid (Clariant) | 30.00% |
| | Sodium Laureth Sulfate | |
| | Hostapon ® CGN (Clariant) | 5.00% |
| | Sodium Cocoyl Glutamate | |
| | perfume | 0.30% |
| B | water | ad 100.00% |
| C | phosphoric ester of Example 2 | 1.50% |
| | preservative | q.s. |
| | dye | q.s. |
| | Genagen ® CAB (Clariant) | 8.00% |
| | Cocamidopropyl Betaine | |

Preparation:
I Dissolve C in B with stirring and heating to 50° C.
II A little at a time stir the components of A into I.
III If necessary, adjust the pH.

### Formulation Example 10

### Foam Bath

| | | |
|---|---|---|
| A | Genapol ® LRO liquid (Clariant) | 60.00% |
| | Sodium Laureth Sulfate (30% by weight active content) | |
| B | Medialan ® LD (Clariant) | 8.00% |
| | Sodium Lauroyl Sarcosinate | |
| | perfume | 1.50% |
| | Velsan ® CG 070 (Clariant) | 5.00% |
| | PEG-7 Glyceryl Cocoate | |
| C | phosphoric ester of Example 2 | 1.00% |
| D | water | ad 100% |
| E | dye | q.s. |
| | preservative | q.s. |
| | Genagen ® CAB (Clariant) | 6.00% |
| | Cocamidopropyl Betaine | |

Preparation:
I A little at a time stir the components of B into A.
II Dissolve C in D with stirring and heating at 50° C.
III Add I to II.
VI Stir E into III.
V If necessary, adjust the pH.

### Formulation Example 11

### O/W Skin Milk

| | | |
|---|---|---|
| A | Hostacerin ® DGI (Clariant) | 2.00% |
| | Polyglyceryl-2 Sesquiisostearate | |
| | Isopropyl palmitate | 4.00% |
| | Octyldodecanol | 4.00% |
| | Nipaguard ® PDU (Clariant) | q.s. |
| | Propylene Glycol (and) Diazolidinyl Urea (and) | |
| | Methylparaben (and) Propylparaben | |
| B | Aristoflex ® AVC (Clariant) | 1.20% |
| | Ammonium Acryloyldimethyltaurate/VP Copolymer | |
| C | Hostapon ® CGN (Clariant) | 0.60% |
| | Sodium Cocoyl Glutamate | |
| | water | ad 100% |
| D | phosphoric ester of Example 1 | 1.00% |
| E | perfume | 0.40% |

Preparation:
I Dissolve D in C with stirring and heating to 50° C.
II Add B to A, then add I and stir thoroughly.
III Add E to II.
IV Finally homogenize the formulation.

### Formulation Example 12

### Antiperspirant Roll-On

| | | |
|---|---|---|
| A | phosphoric ester of Example 2 | 1.50% |
| B | water | ad 100.00% |
| C | Locron ® L (Clariant) | 20.00% |
| | Aluminum Chlorohydrate | |
| D | Genapol ® T 250 (Clariant) | 5.00% |
| | Ceteareth-25 | |
| | Butylene Glycol | 3.00% |
| | Cetiol OE | 1.00% |
| | Dicaprylyl Ether | |
| | Glyceryl Isostearate | 2.00% |
| E | SilCare ® Silicone SEA (Clariant) | 0.50% |
| | Trideceth-9 PG Amodimethicone and Trideceth-12 | |

Preparation:
I Dissolve A in B with stirring and heating to 60° C.
II Add C to I.
III Melt D at 50° C. and add II and stir until a clear solution has formed.
IV Add E at 30° C.

Formulation Example 12 displayed a distinct reduction of white residues on the clothing after use of the roll-on on the skin compared with the same formulation but without phosphoric ester according to Example 2.

### Formulation Example 13

### Vitamin C Gel

| | | |
|---|---|---|
| A | phosphoric ester of Example 2 | 1.00% |
| | Genapol ® T 250 (Clariant) | 2.00% |
| | Ceteareth-25 | |
| B | water | ad 100.00% |
| C | ascorbic acid | 3.00% |
| D | Aristoflex AVC (Ciariant) | 0.80% |
| | Ammonium Acryloyldimethyltaurate/VP Copolymer | |

Preparation:
I Dissolve A in B with stirring at 50° C.
II Stir C into I at room temperature.
III Add D and stir until a homogeneous gel has formed.

### Formulation Example 14

### Shower Bath

| | | |
|---|---|---|
| A | phosphoric ester of Example 2 (Clariant) | 3.00% |
| | Aristoflex ® PEA (Clariant) | 2.00% |
| | Polypropylene-Terephthalate | |
| B | water | ad 100% |
| C | Genapol ® LRO liquid (Clariant) | 30.00% |
| | Sodium Laureth Sulfate | |
| | Genapol ® LA 030 (Clariant) | 1.50% |
| | Laureth-3 | |
| | Hostapon ® CLG (Clariant) | 5.00% |
| | Sodium Lauroyl Glutamate | |
| | Genagen ® KB (Clariant) | 6.00% |
| | Coco Betaine | |
| | perfume | 0.30% |
| | dye | q.s. |
| | preservative | q.s. |

Preparation:
I Dissolve A in B at 50° C.
II Stir the components of C into I in succession.
III Adjust pH if necessary.

### Formulation Example 15

### Facial Anti-Aging Cream Gel

| | | |
|---|---|---|
| A | phosphoric ester of Example 1 (Clariant) | 1.00% |
| B | water | ad 100.00% |
| C | paraffin oil | 5.00% |
| | SilCare ® Silicone 31M50 (Clariant) | 3.00% |
| | Caprylyl Trimethicone | |
| D | Aristoflex ® AVC (Clariant) | 1.80% |
| | Ammonium Acryloyldimethyltaurate/VP Copolymer | |
| E | glycolic acid 30%* | 6.00% |
| | Phenonip ® (Clariant) | 0.50% |
| | Phenoxyethanol (and) Methylparaben (and) | |
| | Ethylparaben (and) Butylparaben (and) | |
| | Propylparaben (and) Isobutylparaben | |
| F | Genapol ® LA 070 (Clariant) | 2.00% |
| | Laureth-7 | |

*neutralized to pH 4 with NaOH.

Preparation:
I Dissolve A in B with stirring at 50° C.
II Stir D into C.
III Stir I into II and stir until a homogeneous gel has formed.
IV Add E into III.
V Stir F into IV and stir until cream gel is homogeneous.

### Formulation Example 16

### O/W Self-Tanning Cream

| | | |
|---|---|---|
| A | Hostaphat ® CC 100 (Clariant) | 1.0% |
| | Cetyl Phosphate | |
| | Glyceryl Stearate | 0.5% |
| | Cetearyl Alcohol | 0.5% |
| | paraffin oil | 8.0% |
| | isopropyl palmitate | 7.0% |
| | SilCare ® Silicone 41M15 (Clariant) | 1.0% |
| | Caprylyl Methicone | |
| B | Aristoflex ® AVC (Clariant) | 1.2% |
| | Ammonium Acryloyldimethyltaurate/VP Copolymer | |
| C | water | ad 100% |
| D | phosphoric ester of Example 1 (Clariant) | 1.0% |
| E | Hostapon ® CLG (Clariant) | 0.5% |
| | Sodium Lauroyl Glutamate | |
| | glycerol | 5.0% |
| F | Tocopheryl Acetate | 1.0% |
| | Fragrance | 0.2% |
| | Preservative | q.s. |
| G | Dihydroxyacetone | 5.0% |
| H | water | 8.0% |
| I | sodium hydroxide (10% in water) | q.s. |

Preparation:
I Melt A at 80° C.
II Stir B into A.
III Dissolve D in C at 50° C., then add E.
IV Stir III into II.
V Add F at room temperature.
VI Dissolve G in H and stir into V.
VII Adjust pH to 4-5 with I if necessary.

### Formulation Example 17

### O/W Sunscreen Milk

| | | |
|---|---|---|
| A | Hostaphat ® CK 100 (Clariant) | 2.00% |
| | Potassium Cetyl Phosphate | |
| | SilCare ® Silicone 41M15 (Clariant) | 1.00% |
| | Caprylyl Methicone | |
| | stearic acid | 0.50% |
| | Cetyl Alcohol | 0.50% |
| | Cutina ® GMS | 1.00% |
| | Glyceryl Stearate | |
| | Cetiol ® SN | 4.00% |
| | Cetearyl Isononanoate | |
| | Velsan ® CCT (Clariant) | 4.00% |
| | Caprylic/Capric Triglyceride | |
| | Neo ® Heliopan BB | 1.50% |
| | Benzophenone-3 | |
| | Eusolex ® 6300 | 4.00% |
| | 4-Methylbenzylidene Camphor | |
| B | Aristoflex ® AVC (Clariant) | 0.40% |
| | Ammonium Acryloyldimethyltaurate/VP Copolymer | |
| C | water | ad 100% |
| D | phosphoric ester of Example 2 (Clariant) | 1.00% |
| E | glycerol | 3.00% |
| | Eusolex ® 232 | 2.00% |
| | Phenylbenzimidazole Sulfonic Acid | |
| | Tris(hydroxymethyl)aminomethane | 1.10% |
| | Tromethamine | |
| F | Tocopheryl Acetate | 0.50% |
| | Phenonip ® (Clariant) | 0.50% |
| | Phenoxyethanol (and) Methylparaben | |
| | (and) Butylparaben (and) Ethylparaben | |
| | (and) Propylparaben | |
| | Fragrance | 0.40% |

Preparation:
I Melt A at 80° C., then add B.
II Dissolve D in C at 60° C.
III Stir E into II.
IV Dissolve III in I.
V Add F into IV at 35° C.

### Formulation Example 18

### Facial Anti-Aging Gel

| | | |
|---|---|---|
| A | Genapol ® T 250 (Clariant) | 1.00% |
| | Ceteareth-25 | |
| | phosphoric ester of Example 2 (Clariant) | 1.00% |
| B | water | ad 100% |
| C | Aristoflex ® AVC (Clariant) | 2.00% |
| | Ammonium Acryloyldimethyltaurate/VP Copolymer | |
| D | Glycolic acid 30%* | 6.00% |
| | Phenonip ® (Clariant) | 0.50% |
| | Phenoxyethanol (and) Methylparaben (and) | |
| | Ethylparaben (and) Butylparaben (and) | |
| | Propylparaben (and) Isobutylparaben | |

*neutralized to pH 4 with NaOH.

Preparation:
I Dissolve A in B with stirring at 50° C.
II Add C and stir until a homogeneous gel has formed.
III Add D and stir until the gel is again homogeneous.

### Formulation Example 19

### Hair Shampoo

| | | |
|---|---|---|
| A | Genapol ® LRO liquid (Clariant) | 30.00% |
| | Sodium Laureth Sulfate | |
| | Hostapon ® CGN (Clariant) | 5.00% |
| | Sodium Cocoyl Glutamate | |
| | perfume | 0.30% |
| B | water | ad 100.00% |
| C | phosphoric ester of Example 2 | 1.50% |
| | benzoic acid | 0.50% |
| | dye | q.s. |
| | Genagen ® CAB (Clariant) | 8.00% |
| | Cocamidopropyl Betaine | |

Preparation:
I Dissolve C in B with stirring and heating to 50° C.
II A little at a time stir the components of A into I.
II Adjust the pH to 4.9.

### Formulation Example 20

### Foam Bath

| | | |
|---|---|---|
| A | Genapol ® LRO liquid (Clariant) | 60.00% |
| | Sodium Laureth Sulfate (30% by weight active content) | |
| B | Medialan ® LD (Clariant) | 8.00% |
| | Sodium Lauroyl Sarcosinate | |
| | perfume | 1.50% |
| | Velsan ® CG 070 (Clariant) | 5.00% |
| | PEG-7 Glyceryl Cocoate | |
| C | phosphoric ester of Example 2 | 1.00% |
| D | water | ad 100% |
| E | dye | q.s. |
| | sorbic acid | 0.60% |
| | Genagen ® CAB (Clariant) | 6.00% |
| | Cocamidopropyl Betaine | |

Preparation:
I A little at a time stir the components of B into A.
II Dissolve C in D with stirring and heating to 50° C.
III Add Ito II.
IV Stir E into III.
V Adjust the pH to 4.8.

### Formulation Example 21

### Shower Bath

| | | |
|---|---|---|
| A | phosphoric ester of Example 2 (Clariant) | 3.00% |
| | Aristoflex ® PEA (Clariant) | 2.00% |
| | Polypropylene Terephthalate | |
| B | water | ad 100% |
| C | Genapol ® LRO liquid (Clariant) | 30.00% |
| | Sodium Laureth Sulfate | |
| | Genapol ® LA 030 (Clariant) | 1.50% |
| | Laureth-3 | |
| | Hostapon ® CLG (Clariant) | 5.00% |
| | Sodium Lauroyl Glutamate | |
| | Genagen ® KB (Clariant) | 6.00% |
| | Coco Betaine | |
| | perfume | 0.30% |
| | dye | q.s. |
| | sodium salicylate | 0.50% |

Preparation:
I Dissolve A in B at 50° C.
II Stir the components of C into I in succession.
III Adjust the pH to 4.5.

1. A composition comprising
A) at least one phosphoric ester of formula (I) 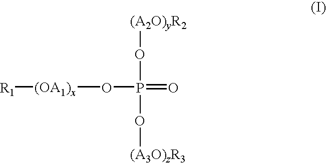
where
R₁, R₂ and R₃ may be identical or different and each is a linear or branched saturated alkyl group having 6 to 30, carbon atoms, is a linear or branched mono- or polyunsaturated alkenyl group having 6 to 30, carbon atoms, or is an aryl group, more particularly a phenyl group, which may be substituted with 1 to 3 branched alkyl groups which each independently comprise 3 to 18 carbon atoms,
the individual groups (OA₁)ₓ, (A₂O)_{y} and (A₃O), each independently consist of units selected from the group consisting of CH₂CH₂O, C₃H₆O and C₄H₈O, and the units CH₂CH₂O, C₃H₆O and C₄H₈O may be arranged blocklike or randomly distributed within the individual groups (OA₁)ₓ, (A₂O)_{y} and (A₃O)_{z}, and
x, y and z are each independently a number from 30 to 150, and
B) water in an amount >50.0% by weight, based on the final composition.

2. A composition according to claim 1, wherein R₁, R₂ and R₃ in the phosphoric esters of formula (I) may be identical or different and each is a linear or branched saturated alkyl group having 6 to 30, carbon atoms, or is a linear or branched mono- or polyunsaturated alkenyl group having 6 to 30, carbon atoms.

3. A composition according to claim 1, wherein OA₁, OA₂ and OA₃ in the phosphoric esters of formula (I) are each CH₂CH₂O.

4. A composition according to claim 1, wherein R₁—(OA₁)ₓ, R₂—(OA₂)_{y} and R₃—(OA₃)_{z} in the phosphoric esters of formula (I) are derived from C_{16/18} fatty alcohol ethoxylates having 30 to 150 ethylene oxide units.

5. A The-composition according to claim 1, further comprising at least one phosphoric ester of formula (II) 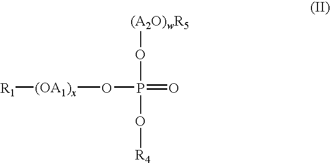
wherein
R₁ is a linear or branched saturated alkyl group having 6 to 30, carbon atoms, is a linear or branched mono- or polyunsaturated alkenyl group having 6 to 30, carbon atoms, or is an aryl group which may be substituted with 1 to 3 branched alkyl groups which each independently comprise 3 to 18 carbon atoms,
R₄ is H, Li⁺, Na⁺, K⁺, Mg⁺⁺, Ca⁺⁺, Al⁺⁺⁺, NH₄⁺ or quaternary ammonium ions [HNR^{a}R^{b}R^{c}], in which R^{a}, R^{b} and R^{c} are independently stand for hydrogen, a linear or branched alkyl group having 1 to 22 carbon atoms, a linear or branched mono- or polyunsaturated alkenyl group having 2 to 22 carbon atoms, a linear monohydroxyalkyl group having 2 to 10 carbon atoms, or a linear or branched dihydroxyalkyl group having 3 to 10 carbon atoms,
R₅ has the meaning of R₁ or R₄,
the individual groups (OA₁)ₓ and (A₂O)_{w} each independently consist of units selected from the group consisting of CH₂CH₂O, C₃H_{S}O and C₄H₈O and the units CH₂CH₂O, C₃H₆O and C₄H₃O may be arranged blocklike or randomly distributed within the individual groups (OA₁)ₓ and (A₂O)_{w},
x is a number from 30 to 150, and
w is 0 or is a number from 30 to 150,
with the proviso that the amount of phosphoric triesters as per formula (I) is greater than 80.0% by weight, based on the total weight of the phosphoric esters as per formula (I) and formula (II), and the degree of neutralization of the nonesterified phosphorus valences (P—OH) in the phosphoric esters as per formula (II) may be between 0 and 100%.

6. A composition according to claim 5, comprising at least one compound of formula (II) where R₅ has the meaning of R₁ and w is a number from 30 to 150.

7. A composition according to claim 1, comprising water in an amount >90.0% by weight based on the final composition.

8. A composition according to claim 1, comprising the at least one phosphoric ester of formula (I) in an amount of 0.1% to 10.0% by weight, based on the final composition.

9. A composition according to claim 1, further comprising at least one surfactant.

10. A composition according to claim 9 comprising the at least one or phosphoric ester of formula (I) in an amount of 0.1% to 5.0% by weight, based on the final composition.

11. A cosmetic, pharmaceutical or dermatological composition comprising at least one composition according to claim 1.

12. A composition according to claim 1, wherein the composition is in the form of an aqueous, aqueous-alcoholic or aqueous-surfactant composition, in the form of an emulsion, in the form of a suspension, in the form of a dispersion or in the form of a spray.

13. A composition according to claim 1 having a pH in the range from 2 to 10.

14. A composition according to claim 1 having a pH in the range from 2.5 to 5 further comprising, based on the entire composition, from 0.05% to 3.0% by weight of at least one organic acid having an antimicrobial effect.

15. A composition according to claim 1 further comprising at least one electrolyte.

16. A composition according to claim 15 wherein the amount of the at least one electrolyte is from 0.1% to 20.0% by weight, based on the final composition.

17. A composition according to claim 1, further comprising hydrogen peroxide or hydrogen peroxide releasers.

18. A deodorant or antiperspirant formulation comprising a composition according to claim 1.

19. A method for reducing the formation of white residues on the clothing after using the deodorant or antiperspirant formulations on the skin comprising the step of contacting the skin with a composition according to claim 18.

20. A thickener, consistency regulator, emulsifier, sensory additive, solubilizer, dispersant, glidant, adhesive or stabilizer comprising a composition according to claim 1.

21. (canceled)

22. A composition according to claim 12, wherein the composition is the form of an aqueous, aqueous-alcoholic or aqueous-surfactant composition.
